(19) United States
(12) Patent Application Publication
(10) Pub. No.: US 2017/0232133 A1
(43) Pub. Date: Aug. 17, 2017

(54) Articles and Method for Improved Transfer of Bodily Fluids

(71) Applicant: TamiCare Ltd., Heywood (GB)

(72) Inventor: Tamar Giloh, Heywood (GB)

(21) Appl. No.: 15/433,267

(22) Filed: Feb. 15, 2017

Related U.S. Application Data
(60) Provisional application No. 62/295,744, filed on Feb. 16, 2016.

(51) Int. Cl. A61L 15/18; A61F 13/531; A61F 13/538; A41B 1/08; D06M 11/74; A43B 1/00; A41C 3/00; A43B 5/00; B08B 1/00; A61F 13/00; A41D 3/00

(52) U.S. Cl. CPC A61L 15/18; A61F 13/00068; A61F 13/531; A61F 13/538; A41B 1/08; A41D 3/00; A43B 1/00; A41C 3/005; A43B 5/00; B08B 1/006; D06M 11/74; A61F 2013/15146

(57) ABSTRACT

A method is provided to accelerate transfer of released body fluids from a body through a treated textile fabric to promote dryness of a skin of the body, the method comprising providing a treated textile fabric facing the body fluids to be transferred, wherein the treated textile fabric includes a first surface and a second surface, wherein the first surface is positioned facing towards the body fluids and the second surface is positioned facing away from the body fluids, wherein the treated textile fabric is structured with a plurality of pores so to allow a passage of a liquid from the first surface to the second surface, and wherein the first surface is coated with a graphene material such that a permeation flux of the first surface is greater than a permeation flux of the second surface.

### RELATED APPLICATIONS

This application claims priority to U.S. Provisional Application No. 62/295,744 entitled “Articles and Method for Improved Transfer of Bodily Fluids,” filed on Feb. 16, 2016, which is hereby incorporated by reference in its entirety.

### BACKGROUND OF THE INVENTION

Hygiene absorbing products, such as diapers and pads, which are mostly made of different nonwoven fabrics, are widely used to capture bodily fluids, remove these fluids from the contact area with the body skin, and/or keep those fluids away of the user skin as much as possible. Such hygiene absorbing products are design to accomplish these goals even under body pressure, such as when the individual wearing such product is in a sitting position, to promote skin dryness. Therefore, challenges involving such products are mainly related to the speed of absorption and the lowest possible wet-back level under pressure. Hygiene products brands, such as Always®, Kotex®, Tena®, Huggies®, Pampers®, and other nonwoven producers compete on improving the above-mentioned performance.

Medical articles, such as medical bandages and specialty pads, made of both nonwoven fabrics and textile fabrics, which are mainly used for wound care, require high level performance of bodily menses absorption. All of these body menses are basically made of water. The goals of such products are quite similar to those described above: absorption speed with low wet-back and fluids distribution, with the main difference of those fluids being, in many cases, of higher viscosity, thus creating the quick absorption very hard to achieve, negatively affecting speed of wound healing.

While both hygiene and medical products, as described above, are meant to absorb bodily fluids, there is a desire in textile clothing to transfer body fluids, such as sweat, away from the garment surface which is facing the user skin, to a middle layer or to the outer surface of the clothing fabric. Speed of sweat transfer is important for the improvement of comfort, while wearing the article of clothing, especially while engaged in sports activities which might generate high levels of sweat, resulting in negative impact on performance of the person wearing the article.

### SUMMARY OF THE INVENTION

The current invention discloses a method to improve all the different products described above, while using a surprisingly cost effective solution. In particular, the present invention relates to a method to significantly improve the transfer of body fluids, such as blood, urine, sweat and the like, away from the body, for example a human body, to or through a fabric which is in close contact with the body. The present invention also relates to articles produced by the method.

The above mentioned improvement is achieved by coating said fabrics with graphene-based material, such as but not limited to graphene oxide (GO) and reduced graphene oxide (rGO). Graphene, a single layer of graphite, has been considered as a mostly suitable material for creation of membranes as it is extremely thin, therefore, providing high permeation flux. Graphene-based membranes, of different structures and thicknesses, also improve water/foul separation.

The inventors found that applying graphene based water dispersions, (such as those disclosed in US Patent Application Pub. No. 2016/0310908, which is hereby incorporated by reference), as a coating on different non-woven and textile fabrics, having built-in porosity (mainly by their nature), results in significantly improved absorption speed and moisture transfer performance. The fabric may be any woven, non-woven, or fabric-like product which is used close to the body. The fabric may be disposable or reusable. For example, the fabric may be an absorption pad, such as but not limited to hygiene pads, for example, feminine hygiene pads, adult incontinence pads and diapers, baby diapers and the like; any other garment and clothing, for example, undergarment, shirts, bras and bras components; shoes and sport shoes and their components; medical bandages, cosmetic bandages, masks and any other desired body touching product.

In another example, the invention comprises a method including a step of coating, by spraying, brushing, printing or by any other suitable method, at least part of the fabric with graphene, graphene oxide (GO), reduced graphene oxide (rGO), or any other graphene derived material, preferably in a liquid state such as, but not limited to, a suspension. The spraying, brushing, printing of the suspension onto the fabric creates a membrane on the fabric.

Thus, in a first aspect, the invention comprises a method to accelerate transfer of released body fluids from a body through a treated textile fabric to promote dryness of a skin of the body, the method comprising providing the treated textile fabric facing the body fluids to be transferred, wherein the treated textile includes a first surface and a second surface, wherein the first surface is positioned facing towards the body fluids and the second surface is positioned facing away from the body fluids, wherein the treated textile is structured with a plurality of pores so to allow a passage of a liquid from the first surface to the second surface, and wherein the first surface is coated with a graphene material such that a permeation flux of the first surface is greater than a permeation flux of the second surface.

In one embodiment, said treated textile fabric constitutes a part of a product.

In another embodiment, the body fluids are transferred from one surface of the textile fabric to the second surface, kept on the second surface of the treated textile fabric or released to the open air.

In another embodiment, the body fluids are transferred and absorbed by other components of said product.

In another embodiment, said treated textile fabric is a part of hygiene absorption product, such as hygiene pad or diaper.

In another embodiment, said treated textile fabric is a part of a shoe.

In another embodiment, said treated textile fabric is a part of a clothing product, such as shirt or coat.

In another embodiment, said treated textile fabric is a wipe.

In another embodiment, the coated first surface promotes and accelerates transfer of body liquids from the first surface to the second surface at a speed higher than the speed of liquid transfer through the same textile fabric before said treatment.

In another embodiment, said treated textile fabric acts as a filter which improves separation of water from solids suspended or dissolved within the body fluids.

In another embodiment, the graphene material is graphene oxide (GO) or reduced graphene oxide (rGO).

In another embodiment, the treated textile fabric is a nonwoven fabric.

In a second aspect, the invention comprises a method comprising (a) dispersing a graphene material in a solvent to form a dispersion, and (b) applying the dispersion on at least a portion of a first surface of a textile fabric to form a graphene membrane, wherein the first surface is configured to be positioned facing towards a body of a user and the second surface is configured to be positioned facing away from the body of the user, wherein the membrane is structured with a plurality of pores to allow a passage of a liquid from the first surface to the second surface, and wherein the graphene membrane causes a permeation flux of at least a portion of the first surface to be greater than a permeation flux of the second surface.

In one embodiment, applying the dispersion on at least a portion of a first surface of the textile fabric comprises coating, spraying, brushing, or printing the dispersion.

In a third aspect, the invention comprises a treated textile fabric comprising (a) a textile fabric having a first surface and a second surface, and (b) a graphene membrane on at least a portion of the first surface of the textile fabric, wherein the first surface is configured to be positioned facing towards a body of a user and the second surface is configured to be positioned facing away from the body of the user, wherein the membrane is structured with a plurality of pores so to allow a passage of a liquid from the first surface to the second surface, and wherein a permeation flux of at least a portion of the first surface is greater than a permeation flux of the second surface.

These as well as other aspects, advantages, and alternatives, will become apparent to those of ordinary skill in the art by reading the following detailed description,

### DETAILED DESCRIPTION OF THE INVENTION

Exemplary articles and methods are described herein. It should be understood that the word “exemplary” is used herein to mean “serving as an example, instance, or illustration.” Any embodiment or feature described herein as “exemplary” is not necessarily to be construed as preferred or advantageous over other embodiments or features. The exemplary embodiments described herein are not meant to be limiting. It will be readily understood that certain aspects of the disclosed articles and methods can be arranged and combined in a wide variety of different configurations, all of which are contemplated herein.

As used herein, “permeation flux” means the volume flowing through a membrane per unit area per unit time.

As used herein, representative “products” include an absorption pad, such as but not limited to hygiene pads such as feminine hygiene pads, adult incontinence pads and diapers, baby diapers and the like; any other garment and clothing, for example, undergarment, shirts, bras and bras components; shoes and sport shoes and their components; medical bandages, wound care products, cosmetic bandages, masks and any other desired body touching product.

As described above, the graphene material coating treatment of one or more layers on the textile porous surface promotes the creation of hydrophilic structures on or within the textile fabric by creating a series of hydrophilic channels within the material pores, resulting in superior permeation flux. Surprisingly, water passes through these treated pores faster than it would pass through the same pores of a non-treated textile fabric.

By treating a first surface of a textile fabric with the graphene material and not treating the second, opposite surface, a permeation flux of the first surface may be greater than a permeation flux of the second surface, enabling water to pass through these treated textile fabrics faster than it would pass through the same non-treated textile fabric.

In addition, the treated fabric acts as a filter which improves not only the water flux, but also the separation of water from other components normally suspended or dissolved within the body fluids, such as proteins, minerals, salts, particles, solids, etc. and where the water is quickly absorbed by absorption/distribution fabrics, while the non-water dissolved components are left on top of the fabric and are either absorbed in a slower manner by other mechanisms or by other functional layers, or left on the fabric to be removed later. An example for such later removal could be washing the product for a reused product, or throwing it away in the case of a disposable product. As a result, the wetting properties and the wicking of fabric are largely improved which, in turn, surprisingly enhances the ability of the fabric to transfer greater amount of body fluids at a given time.

Reference now will be made to the embodiments of the invention, one or more examples of which are set forth below.

In one embodiment, the treated textile is a part of hygiene absorption product, such as hygiene pad or diaper; wherein an improved disposable hygiene pad is provided, the pad comprising a top-sheet, a wicking/distribution layer, an absorption layer and a back-sheet. The pad also comprises at least part of one of the pad layers coated with a GO liquid coating and dried. The coating of the nonwoven sheet layer may be done in different areas, with different coating structures. For example, the coating of the first layer could be done in the core area of the product, while the next layer could be coated in other areas of the improved water distribution along the pad in a more controllable manner.

In another embodiment, any of the pad layers may be coated with a GO liquid coating.

In yet another embodiment, the body fluids are transferred and absorbed by other components of said product, for example, the coated textile can be placed as a middle layer of a hygiene pad, or diaper, acting as a separating membrane which accelerates water transfer while leaving other body fluids' physical particles set aside to be treated separately, so that the obstacles and delay that such particles impose on absorption speed is decreased.

In yet another embodiment, the treated textile constitutes part of a clothing product; such product can be a shirt, where the treated part is the inner side of the shirt, usually exposed to more sweating.

In yet another embodiment, the body fluids are transferred from one surface of the textile to the second surface kept on the second surface of the treated textile or released to the open air.

One example of this embodiment is a coat with several interlining layers. Additional examples include socks and shoes provided with the coating.

In yet another embodiment, the coated treated textile is a part of a medical wound care article, wherein the coated textile accelerates removing wound exudates promoting dryness of the wound area.

In another embodiment, the product is a wipe.

The present invention also relates to articles produced by the method.

While various aspects and embodiments have been disclosed herein, other aspects and embodiments will be apparent to those skilled in the art. All embodiments within and between different aspects of the invention can be combined unless the context clearly dictates otherwise.

The various aspects and embodiments disclosed herein are for purposes of illustration and are not intended to be limiting, with the true scope and spirit being indicated by the following claims.

1. A method to accelerate transfer of released body fluids from a body through a treated textile fabric to promote dryness of a skin of the body, the method comprising:
providing the treated textile fabric facing the body fluids to be transferred, wherein the treated textile includes a first surface and a second surface, wherein the first surface is positioned facing towards the body fluids and the second surface is positioned facing away from the body fluids, wherein the treated textile is structured with a plurality of pores so to allow a passage of a liquid from the first surface to the second surface, and wherein the first surface is coated with a graphene material such that a permeation flux of the first surface is greater than a permeation flux of the second surface.

2. The method of claim 1, wherein said treated textile fabric constitutes a part of a product.

3. The method of claim 1, wherein the body fluids are transferred from one surface of the textile fabric to the second surface, kept on the second surface of the treated textile fabric or released to the open air.

4. The method of claim 2, wherein the body fluids are transferred and absorbed by other components of said product.

5. The method of claim 4, wherein said treated textile fabric is a part of hygiene absorption product, such as hygiene pad or diaper.

6. The method of claim 2, wherein said treated textile fabric is a part of a shoe.

7. The method of claim 2, wherein said treated textile fabric is a part of a clothing product, such as shirt or coat.

8. The method of claim 2, wherein said treated textile fabric is a wipe.

9. The method of claim 1, wherein the coated first surface promotes and accelerates transfer of body liquids from the first surface to the second surface at a speed higher than the speed of liquid transfer through the same textile fabric before said treatment.

10. The method of claim 1, wherein said treated textile fabric acts as a filter which improves separation of water from solids suspended or dissolved within the body fluids.

11. The method of claim 1, wherein the graphene material is graphene oxide (GO) or reduced graphene oxide (rGO).

12. The method of claim 1, wherein the treated textile fabric is a nonwoven fabric.

13. A method, comprising:
dispersing a graphene material in a solvent to form a dispersion;
applying the dispersion on at least a portion of a first surface of a textile fabric to form a graphene membrane, wherein the first surface is configured to be positioned facing towards a body of a user and the second surface is configured to be positioned facing away from the body of the user, wherein the membrane is structured with a plurality of pores to allow a passage of a liquid from the first surface to the second surface, and wherein the graphene membrane causes a permeation flux of at least a portion of the first surface to be greater than a permeation flux of the second surface.

14. The method of claim 13, wherein applying the dispersion on at least a portion of a first surface of the textile fabric comprises coating, spraying, brushing, or printing the dispersion.

15. A treated textile fabric, comprising:
a textile fabric having a first surface and a second surface; and
a graphene membrane on at least a portion of the first surface of the textile fabric, wherein the first surface is configured to be positioned facing towards a body of a user and the second surface is configured to be positioned facing away from the body of the user, wherein the membrane is structured with a plurality of pores so to allow a passage of a liquid from the first surface to the second surface, and wherein a permeation flux of at least a portion of the first surface is greater than a permeation flux of the second surface.
